(19) United States
(12) Patent Application Publication
(10) Pub. No.: US 2023/0320711 A1
(43) Pub. Date: Oct. 12, 2023

(54) ARTICULATION CONTROL OF FLEXIBLE MEDICAL SYSTEMS

(71) Applicant: Intuitive Surgical Operations, Inc., Sunnyvale, CA (US)

(72) Inventors: Nicola Diolaiti, Menlo Park, CA (US); Sang Gyum Kim, San Jose, CA (US); Samuel B. Schorr, San Jose, CA (US)

(73) Assignee: Intuitive Surgical Operations, Inc., Sunnyvale, CA (US)

(21) Appl. No.: 18/298,495

(22) Filed: Apr. 11, 2023

Related U.S. Application Data
(60) Provisional application No. 63/329,722, filed on Apr. 11, 2022.

(51) Int. Cl. A61B 17/00

(52) U.S. Cl. CPC A61B 17/00234; A61B 2017/00318

(57) ABSTRACT

Methods and systems for controlling articulation of a system are described. In some embodiments, a parameter related to a curvature of one or more portions of an articulable portion of elongated body may be determined. The elongated body may include a channel through which a tool may be inserted. Articulation of the articulable portion of the elongated body may be controlled based at least in part on the determined parameter in some modes of operation including when a tool is moved relative to the channel of the elongated body.

### CROSS-REFERENCE TO RELATED APPLICATIONS

Pursuant to 35 U.S.C. § 119 (e), this application claims benefit of U.S. Provisional Application No. 63/329,722 filed on Apr. 11, 2022, entitled “ARTICULATION CONTROL OF FLEXIBLE MEDICAL SYSTEMS.” The disclosure of the U.S. Provisional Application is incorporated herein by reference in its entirety.

### FIELD

The present disclosure is directed to systems and methods related to articulation control of flexible medical systems.

### BACKGROUND

Minimally invasive medical techniques are intended to reduce the amount of tissue that is damaged during medical procedures, thereby reducing patient recovery time, discomfort, and harmful side effects. Such minimally invasive techniques may be performed through natural orifices in a patient anatomy or through one or more surgical incisions. Through these natural orifices or incisions clinicians may insert minimally invasive medical instruments (including surgical, diagnostic, and/or therapeutic instruments) to reach a target tissue location. One such minimally invasive technique is to use a flexible and/or steerable elongate device, such as a flexible catheter that can be inserted into anatomic passageways and navigated toward a region of interest within the patient anatomy. Medical tools, such as biopsy instruments, may be deployed through the catheter to perform a medical procedure at the region of interest.

### SUMMARY

In one embodiment, a medical system includes: an elongated body including a channel extending through the elongated body, where the elongated body includes an articulable portion extending along at least a portion of a length of the elongated body; one or more actuators operatively coupled to the articulable portion of the elongated body by one or more flexible tethers, wherein the one or more actuators are configured to apply a tension to the one or more flexible tethers to articulate the articulable portion of the elongated body; one or more sensors configured to sense a first parameter related to a shape of the articulable portion of the elongated body; and a processor operatively coupled with the one or more actuators and the one or more sensors. In some embodiments, the processor is configured to: determine a second parameter related to a curvature of one or more portions of the articulable portion based at least in part on the sensed first parameter; and control the one or more actuators based at least in part on the determined second parameter.

In one embodiment, a method for controlling articulation of a medical system includes: determining a parameter related to a curvature of one or more portions of an articulable portion of an elongated body including a channel extending through the elongated body; and controlling articulation of the articulable portion based at least in part on the determined parameter.

In one embodiment, at least one non-transitory computer-readable medium may include instructions thereon that, when executed by at least one processor, perform the above method and/or any other method disclosed herein.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting examples when considered in conjunction with the accompanying figures.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIG. 1 depicts a simplified diagram of one embodiment of a medical system that may include an articulable instrument;

FIG. 2 depicts a simplified diagram of one embodiment of an articulable instrument;

FIG. 3A depicts one embodiment of an articulable elongated body in an articulated configuration;

FIG. 3B depicts the elongated body of FIG. 3A undergoing a displacement during insertion of a tool into a channel of the elongated body;

FIG. 3C depicts the elongated body of FIG. 3A returning to a commanded tip bend angle after insertion of the tool into the channel of the elongated tool body;

FIG. 4 depicts one embodiment of an articulable elongated body going from an initial commanded tip bend angle to a modified tip bend angle after insertion of a tool into a channel of the elongated body;

FIG. 5 depicts one embodiment of an articulable elongated body segmented into a plurality of sequentially located portions for measurement purposes;

FIG. 6 depicts a block diagram of one embodiment of a control algorithm; and

FIG. 7 depicts a flow diagram of a method for controlling articulation of an elongated body of a medical system during insertion, retraction, or other relative movement of a tool within a channel of an articulable portion of the elongated body.

### DETAILED DESCRIPTION

A flexible robotic-assisted medical system, or other flexible medical device, may permit access to the lungs or other organs of a subject via different routes (e.g., through a patient's mouth, trachea, airway, incisions, etc.). During medical procedures performed with these flexible medical systems, one or more tools may be inserted through a lumen, or other channel, formed in a flexible elongated body (also referred to as an “articulatable elongated body” or “elongated body”) which may include an articulable portion. For example, lung biopsy procedures include inserting an elongated, flexible device with an internal channel into a patient's mouth through the airway to a target tissue site (e.g., lesion) in the lungs. A flexible biopsy tool is then inserted through the internal channel of the elongated body to reach a distal opening of the elongated device positioned at the target tissue. When passing the tool through the elongated body to reach the target tissue in either this, or other types of procedures, the tool may be passed through articulated portions of the elongated body of the medical system.

During tool insertion, retraction, or other relative movement of the tool through the lumen, or other internal channel, of a flexible elongated body of a medical system, the tool may vary a stiffness of the combined system of the flexible elongated body and tool. The relative movement of the tool may also apply forces to the flexible elongated body. Due to the flexible elongated body oftentimes being articulated to a desired position and/or orientation, this change in stiffness and additional applied forces may change the position and orientation of the articulated elongated body away from a commanded position and orientation. A control strategy that may be implemented to control articulable systems may include maintaining an end effector at a commanded tip bend angle and/or position. Accordingly, this type of control may oppose the movement of a tool within an articulated elongated body of the medical system as the system may actively control articulation of the elongated body to resist the elongated body deviating from a commanded tip bend angle and/or position. These resulting increased forces associated with the relative movement of a tool through a stiff articulated elongated body (e.g., a catheter or other system exhibiting a tight bend radius) may cause abrasion, cutting, and/or other undesirable damage to the internal surface of the channel, a liner of the channel, and/or the tool during operation.

In view of the above, in some embodiments, a medical system alters the control of the articulation of an elongated body when relative movement between a tool disposed in a channel of the elongated body occurs. Such an operating mode may correspond to a control strategy that permits the articulable portion of the elongated body to deviate from a commanded position and/or orientation during relative movement of the tool within the channel of the elongated body. In some embodiments, holding of the tip position and/or orientation may be weighted against deviation from the commanded tip position and/or orientation during relative movement of the tool to balance holding tip pose while also facilitating tool passage. This may help to reduce the forces applied to the tool and the channel of the elongated body during this relative movement. Such a method may include determining information related to a configuration of the articulable portion of the elongated body and altering the control of the articulable portion the elongated body based at least in part on the information.

In one embodiment, a desired articulation control strategy may include determining a parameter related to a shape of one or more portions of an articulable portion of an elongated body. In some embodiments, the parameter may be related to a radius of curvature of the one or more portions of the articulable portion of the elongated body. As described above, the elongated body may include a channel that is configured to accept one or more tools that are inserted from a proximal portion of the elongated body to a distal end of the elongated body such that the one or more tools may interact with a target tissue. The articulation of the articulable portion may be controlled based at least in part on the determined parameter of the one or more portions of the articulable portion the elongated body. In some embodiments, this control strategy may be implemented when relative movement of the tool and the elongated body occurs, though instances in which the control strategy is implemented when no relative movement between the tool and channel is present are also contemplated.

In one embodiment, and as elaborated on further below, a modified control strategy for an articulable portion of an elongated body during relative movement of a tool and the elongated body may be implemented when one or more threshold parameters are met. For example, the control strategy of the articulable portion may be changed when an average, minimum, or other appropriate characterization of a radius of curvature of one or more portions of the articulable portion the elongated body is less than a corresponding threshold radius of curvature. From a physical standpoint, this may correspond to a radius of curvature of the elongated body that may be smaller than desirable during insertion, retraction, and/or other relative movement of the tool within the elongated body. Other appropriate parameters and thresholds related to a shape of the articulable elongated body are also contemplated.

While any appropriate threshold radius of curvature may be used, in some embodiments, the threshold radius of curvature may be greater than or equal to 4 mm, 5 mm, 6 mm, 7 mm, 8 mm, 9 mm, 10 mm, and/or any other appropriate dimension. The threshold radius of curvature may also be less than or equal to 15 mm, 10 mm, 9 mm, 8 mm, 7 mm, 6 mm, and/or any other appropriate dimension. Combinations of the foregoing are contemplated including, for example, a threshold radius of curvature may be between or equal to 4 mm and 15 mm. This range may correspond to a desirable minimum radius of curvature for many tools inserted through an articulable elongated body for various medical procedures. A threshold radius of curvature may also be greater than or less than the ranges noted above.

In some embodiments, the above-noted control strategy may be operated to increase a radius of curvature of the articulable portion of the elongated body, reduce a tension or other force applied to articulate the elongated body, decrease a commanded tip bend angle, and/or to provide any other desired change in the operation of the articulable portion as elaborated on further below. Accordingly, it should be understood that the currently disclosed methods and systems are not limited to any single type of control during the relative movement of a tool and elongated body the tool is disposed within.

It should be understood that any appropriate type of sensor capable of sensing a parameter related to a position, shape, or curvature of an articulable elongated body may be used in the various embodiments disclosed herein. For example, appropriate sensors may include, but are not limited to, shape sensors including fiber-optic shape sensors, electro-resistive sensors, strain gauges disposed along an elongated body, rotary position sensors, and/or any other type of sensor configured to sense any appropriate parameter related to a position, shape, and/or curvature of the articulable portion the elongated body. This may include, for example, the relative orientations of sequentially located portions of the articulable elongated body which may be used together to determine an overall orientation and/or shape of one or more portions the articulated elongated body. In view of the above, it should be understood that the current disclosure is not limited to any specific type of sensor.

In some embodiments, it may be desirable to provide a sufficient number of sensors disposed along a length of an articulable portion of an elongated body to accurately capture a shape of the elongated body. For example, in some embodiments, the number of sensors may have a spatial frequency that is at least two times a minimum spatial frequency that an associated articulable portion of the elongated body may be positioned in. In some embodiments, this may correspond to 3 to 4 sensors per articulable portion of the elongated body, though other numbers of sensors may also be used. Additionally, in some embodiments, a spacing between the sensors may be greater than or equal to 2 mm, 3 mm, 4 mm, 5 mm, 10 mm, and/or any other appropriate distance. The spacing between the sensors may also be less than or equal to 15 mm, 10 mm, 5 mm, and/or any other appropriate distance. Combinations of foregoing are contemplated including, for example, a spacing between adjacent sensors along a length of an articulable portion of the elongated body that is between or equal to 2 mm and 15 mm.

While specific numbers of sensors per articulable portion, spacings, and spatial frequencies are provided above, it should be understood that the number of sensors, there spacing, and spatial frequencies may be both greater than or less than those noted above. Specifically, a desired spacing and/or spatial frequency of sensors along an articulable portion of elongated body may depend on a number of different parameters including, for example, mechanical properties of the elongated body such as a stiffness of the elongated body which may or may not include a stiffness of the tool to be inserted into the elongated body. Additionally, different portions of an elongated body may exhibit different spatial frequencies for the articulated shape due to different stiffnesses in different portions of the elongated body. Accordingly, a spacing, spatial frequency, and/or number of sensors may change along a length of the elongated body in some embodiments.

It should be understood that any appropriate type of tool that may be inserted through a channel of an articulable elongated body. In some embodiments, the tool may be a medical tool for use in a medical procedure. This may include, but not limited to, biopsy tools, ablation tools, cutting tools, forceps, imaging tools, and/or any other appropriate type of medical tool that may be inserted through a channel of an articulable elongated body.

The articulable elongated bodies disclosed herein may correspond to any appropriate type of flexible elongated body including, for example, flexible shafts, a plurality of sequentially connected links that are pivotally connected to one another to form an overall flexible elongated body, and/or any other appropriate type of articulable elongated body. Additionally, these articulable elongated bodies may be articulated using any appropriate type of articulation method including articulation forces applied to one or more portions of the elongated body using tethers such as cables, wires, tendons, flexible strip, spines, and/or any other tether or other structure capable of transmitting an articulation force to a desired portion of the elongated body. In some embodiments, the applied articulation forces may cause the articulable elongated body to articulate in multiple directions using articulation forces applied using multiple tethers or other structures operatively coupled with the articulable portion of the elongated body. It should also be understood that any appropriate type of actuator capable of applying the desired forces to the tethers or other structures operatively coupled to the articulable portion of the elongated body may be used as described further below.

The disclosed methods and systems may provide a number of benefits depending on the particular application. For example, in some applications, the disclosed methods and systems may result in reduced forces and increased radii of curvature being experienced by a tool during the relative movement of a tool within a channel of an articulated elongated body. This may correspondingly result in reduced damage to both the tools as well as the channel and/or liner positioned within the channel during relative movement of the tool within the channel of the elongated body. In some instances, this may provide both easier tool insertion and retraction as well as longer tool and system life. While several potential benefits are listed above, it should be understood that in some embodiments other benefits different from those noted above may be provided.

As noted above, in some embodiments, the disclosed methods and systems may be used with flexible medical systems such as endoscopes, catheters, and/or any other medical system including an articulable elongated body including a channel that a tool may be moved through. However, while some embodiments provided herein are related to usage of medical systems with robotic-assisted surgical, diagnostic, and/or therapeutic procedures, any reference to medical or surgical instruments as well as medical or surgical methods is non-limiting. Specifically, the systems, instruments, and methods described herein may be used for manual operations, robotic-assisted operations, and/or any other desired usage. Additionally, the systems, instruments, and methods described herein may be used for operations related to humans, animals, human cadavers, animal cadavers, portions of human or animal anatomy, organ models, non-surgical diagnosis, as well as for industrial systems and general robotic, general teleoperational, robotic medical systems, and/or any other appropriate application. Additionally, applications of the currently disclosed systems and methods may be used for non-medical applications as well in some embodiments.

This disclosure describes various instruments and portions of instruments in terms of their state in three-dimensional space. As used herein, the term “position” refers to the location of an object or a portion of an object in a three-dimensional space (e.g., three degrees of translational freedom along Cartesian x-, y-, and z-coordinates). As used herein, the term “orientation” refers to the rotational placement of an object or a portion of an object (three degrees of rotational freedom—e.g., roll, pitch, and yaw). For the sake of clarity, a tip bend angle may refer to an orientation of the distal end portion of an elongated body relative to an orientation of a proximal portion of the elongated body, such as a base or rigid portion of the elongated body that is attached to the articulable portion of the elongated body. As used herein, the term “pose” refers to the position of an object or a portion of an object in at least one degree of translational freedom and to the orientation of that object or portion of the object in at least one degree of rotational freedom (up to six total degrees of freedom). As used herein, the term “shape” refers to a set of poses, positions, or orientations measured along an object.

As used herein, a nominal control strategy for controlling articulation of an articulable portion of a flexible elongated body may correspond to a control strategy that is applied either prior to or after the relative movement of the tool and the elongated body is finished (e.g., prior to and/or after full insertion or removal of the tool relative to the elongated body). For example, in some embodiments, a nominal control strategy may correspond to an orientation control strategy where a bend angle of a distal end of the elongated body is maintained at a commanded bend angle. However, it should be understood that other potential nominal control strategies may also be used prior to and/or after implementation of the control strategies disclosed herein.

Turning to the figures, specific non-limiting examples are described in further detail. It should be understood that the various systems, components, features, and methods described relative to these examples may be used either individually and/or in any desired combination as the disclosure is not limited to only the specific examples described herein.

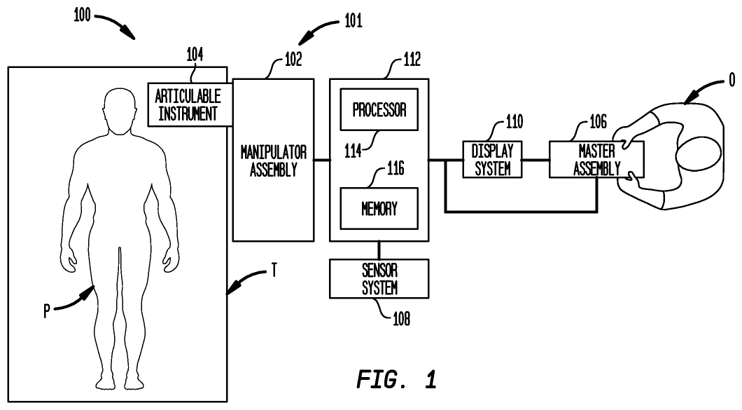

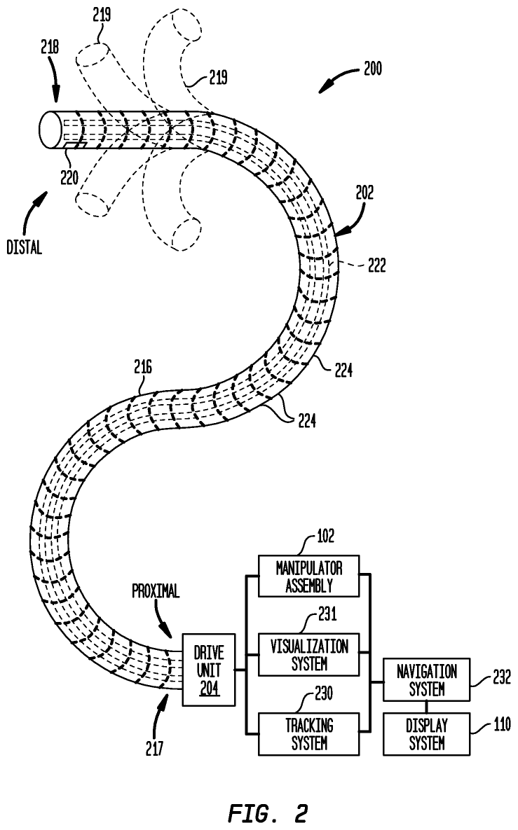

In some examples, the methods and systems disclosed herein may be used in a medical procedure performed with a robotic-assisted medical system as described in further detail below. As shown in FIG. 1, a robotic-assisted medical system 100 may include a manipulator assembly 102 for operating an articulable instrument 104 in performing various procedures on a patient P positioned on a table T in a surgical environment 101. The articulable instrument 104 may correspond to various types of articulable instruments, and may include a flexible elongated body. Additionally or alternatively, the articulable instrument 104 may be a catheter having a lumen or other internal channel, as will be described in further detail with reference to FIG. 2. In these examples, any tool may be inserted into the lumen of the articulable instrument 104. The manipulator assembly 102 may be robotic-assisted, machine operated, manually operated, or a hybrid assembly with select degrees of freedom of motion that may be motorized and/or select degrees of freedom of motion that may be non-motorized. A master assembly 106, which may be inside or outside of the surgical environment 101, generally may include one or more control devices for controlling manipulator assembly 102. Manipulator assembly 102 supports articulable instrument 104 and may include a plurality of actuators or motors that drive inputs on articulable instrument 104 in response to commands from a control system 112. The actuators may include drive systems that when coupled to articulable instrument 104 may advance articulable instrument 104 into a naturally or surgically created anatomic orifice. Other drive systems may move the distal end of the articulable instrument in multiple degrees of freedom, which may include three degrees of linear motion (e.g., linear motion along the X, Y, Z Cartesian axes) and in three degrees of rotational motion (e.g., rotation about the X, Y, Z Cartesian axes). Additionally, the actuators can be used to articulate an articulable portion (e.g., at the distal end) of the articulable instrument 104.

Robotic-assisted medical system 100 also may include a display system 110 for displaying an image or representation of the surgical site and articulable instrument 104 generated by a sensor system 108 which may include an endoscopic imaging system. Display system 110 and master assembly 106 may be oriented so an operator O can control articulable instrument 104 and master assembly 106 with the perception of telepresence. Any of the previously described graphical user interfaces may be displayable on a display system 110 and/or a display system of an independent (e.g., planning) workstation.

In some examples, articulable instrument 104 may include components for use in surgery, biopsy, ablation, illumination, irrigation, or suction. Optionally articulable instrument 104, together with sensor system 108 may be used to gather (e.g., measure or survey) a set of data points corresponding to locations within anatomic passageways of a patient, such as patient P. In some examples, articulable instrument 104 may include components of the imaging system which may include an imaging scope assembly or imaging instrument that records a concurrent or real-time image of a surgical site and provides the image to the operator or operator O through the display system 110. In some examples, imaging system components may be integrally or removably coupled to articulable instrument 104. However, in some examples, a separate endoscope, attached to a separate manipulator assembly may be used with articulable instrument 104 to image the surgical site. The imaging system may be implemented as hardware, firmware, software or a combination thereof which interact with or are otherwise executed by one or more computer processors, which may include the processors of the control system 112.

The sensor system 108 may include a position/location sensor system (e.g., an electromagnetic (EM) sensor system) and/or a shape sensor system for determining the position, orientation, speed, velocity, pose, and/or shape of the articulable instrument 104.

Robotic-assisted medical system 100 may also include control system 112. Control system 112 may include at least one memory 116 and at least one computer processor 114 for effecting control between articulable instrument 104, master assembly 106, sensor system 108, and display system 110. Control system 112 also may include programmed instructions (e.g., a non-transitory machine-readable medium storing the instructions) to implement a plurality of operating modes of the robotic-assisted medical system including a navigation planning mode, a navigation mode, and/or a procedure mode. Control system 112 also may include programmed instructions (e.g., a non-transitory machine-readable medium storing the instructions) to implement some or all of the methods described in accordance with aspects disclosed herein, including, for example, moving a mounting bracket coupled to the manipulator assembly to the connection member, processing sensor information about the mounting bracket and/or connection member, and providing adjustment signals or instructions for adjusting the mounting bracket.

Control system 112 may further include a virtual visualization system to provide navigation assistance to operator O when controlling articulable instrument 104 during an image-guided surgical procedure. Virtual navigation using the virtual visualization system may be based upon reference to an acquired pre-operative or intra-operative dataset of anatomic passageways. The virtual visualization system processes images of the surgical site imaged using imaging technology such as computerized tomography (CT), magnetic resonance imaging (MRI), fluoroscopy, thermography, ultrasound, optical coherence tomography (OCT), thermal imaging, impedance imaging, laser imaging, nanotube X-ray imaging, and/or the like.

FIG. 2 is a simplified diagram of a medical instrument system 200 according to some examples. Medical instrument system 200 may include elongate device 202, which may be the same as or similar to articulable instrument 104 of FIG. 1, coupled to a drive unit 204. Elongate device 202 may include a flexible body 216 having proximal end 217 and distal end 218 (or tip portion 218). Medical instrument system 200 further may include a tracking system 230 for determining the position, orientation, speed, velocity, pose, and/or shape of distal end 218 and/or of one or more segments 224 along flexible body 216 using one or more sensors and/or imaging devices as described in further detail below.

Tracking system 230 may optionally track distal end 218 and/or one or more of the segments 224 using a shape sensor 222, or other appropriate sensor, or sensors, configured to sense one or more parameters related to a shape or curvature of the flexible body 216 of the elongate device 202. Shape sensor 222 may optionally include an optical fiber aligned with flexible body 216 (e.g., provided within an interior channel (not shown) or mounted externally). The optical fiber of shape sensor 222 forms a fiber optic bend sensor for determining the shape of flexible body 216. In one alternative, optical fibers including Fiber Bragg Gratings (FBGs) are used to provide strain measurements in structures in one or more dimensions. Various systems and methods for monitoring the shape and relative position of an optical fiber in three dimensions are described in U.S. patent application Ser. No. 11/180,389 (filed Jul. 13, 2005) (disclosing “Fiber optic position and shape sensing device and method relating thereto”); U.S. patent application Ser. No. 12/047,056 (filed on Jul. 16, 2004) (disclosing “Fiber-optic shape and relative position sensing”); and U.S. Pat. No. 6,389,187 (filed on Jun. 17, 1998) (disclosing “Optical Fibre Bend Sensor”), which are all incorporated by reference herein in their entireties. Sensors in some examples may employ other suitable strain sensing techniques, such as Rayleigh scattering, Raman scattering, Brillouin scattering, and Fluorescence scattering. In some examples, the shape of the elongate device 202 may be determined using other techniques. For example, a history of the distal end pose of flexible body 216 can be used to reconstruct the shape of flexible body 216 over the interval of time. In some examples, tracking system 230 may optionally and/or additionally track distal end 218 using a position sensor system 220. Position sensor system 220 may be a component of an EM sensor system with position sensor system 220 including one or more conductive coils that may be subjected to an externally generated electromagnetic field. Each coil of the EM sensor system then produces an induced electrical signal having characteristics that depend on the position and orientation of the coil relative to the externally generated electromagnetic field. In some examples, position sensor system 220 may be configured and positioned to measure six degrees of freedom, e.g., three position coordinates X, Y, Z and three orientation bend angles indicating pitch, yaw, and roll of a base point or five degrees of freedom, e.g., three position coordinates X, Y, Z and two orientation bend angles indicating pitch and yaw of a base point. Further description of a position sensor system is provided in U.S. Pat. No. 6,380,732 (filed Aug. 11, 1999) (disclosing “Six-Degree of Freedom Tracking System Having a Passive Transponder on the Object Being Tracked”), which is incorporated by reference herein in its entirety.

Flexible body 216 may include a channel sized and shaped to receive a medical instrument. In various examples, any of the tools described above may be inserted through the channel of the flexible body 216. These tools may include, for example, image capture probes, biopsy instruments, laser ablation fibers, and/or other surgical, diagnostic, or therapeutic tools. Medical instruments may be used with an imaging instrument (e.g., an image capture probe) also within flexible body 216. Flexible body 216 may include one or more channels.

Flexible body 216 may also house tethers such as cables, linkages, or other steering controls (not shown) that extend between drive unit 204, which may include one or more actuators, and distal end 218 to controllably bend distal end 218 as shown, for example, by broken dashed line depictions 219 of distal end 218. In some examples, at least four cables are used to provide independent “up-down” steering to control a pitch of distal end 218 and “left-right” steering to control a yaw of distal end 218. Steerable elongate devices are described in detail in U.S. patent application Ser. No. 13/274,208 (filed Oct. 14, 2011) (disclosing “Catheter with Removable Vision Probe”), which is incorporated by reference herein in its entirety.

The information from tracking system 230 may be sent to a navigation system 232 where it is combined with information from visualization system 231 and/or the preoperatively obtained models to provide the operator with real-time position information. In some examples, the real-time position information may be displayed on display system 110 of FIG. 1 for use in the control of medical instrument system 200. In some examples, control system 112 of FIG. 1 may utilize the position information as feedback for positioning medical instrument system 200. Various systems for using fiber optic sensors to register and display a surgical instrument with surgical images are provided in U.S. patent application Ser. No. 13/107,562, filed May 13, 2011, disclosing, “Medical System Providing Dynamic Registration of a Model of an Anatomic Structure for Image-Guided Surgery,” which is incorporated by reference herein in its entirety.

In some examples, medical instrument system 200 may be a robotic-assisted medical system. In some examples, manipulator assembly 102 of FIG. 2 may be replaced by direct operator control. In some examples, the direct operator control may include various handles and operator interfaces for hand-held operation of the instrument.

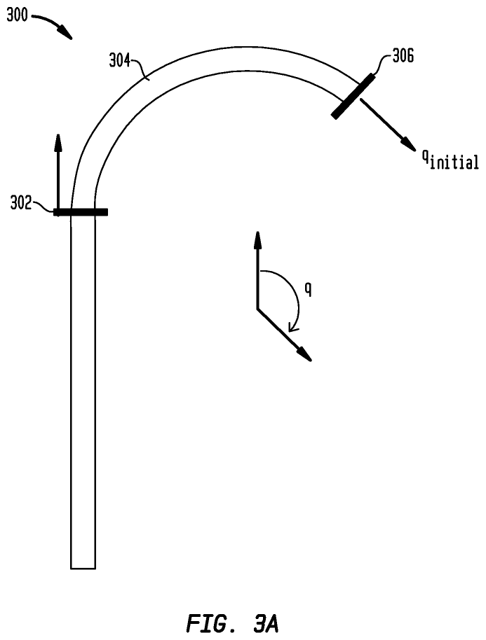

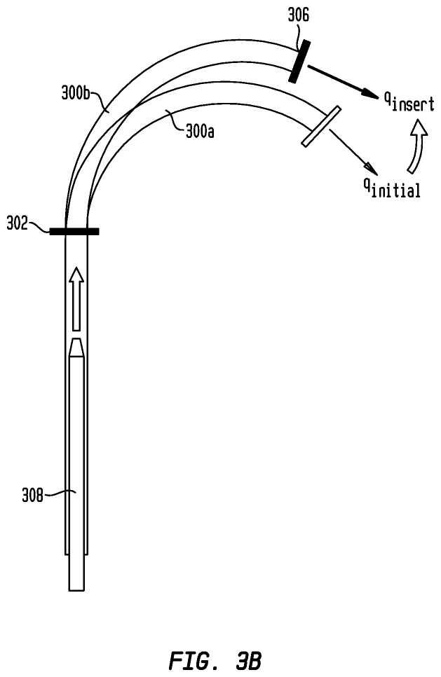

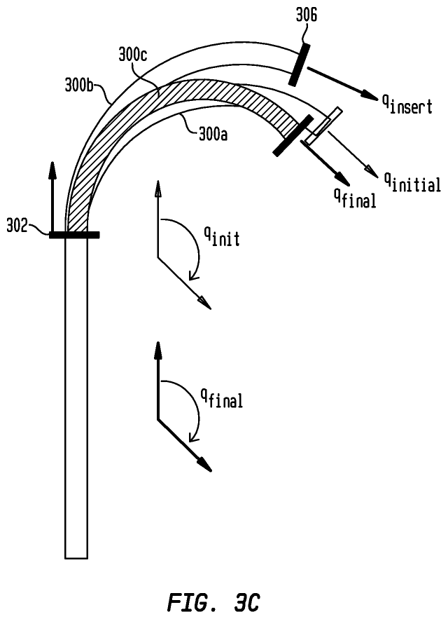

FIG. 3A depicts one embodiment of an elongated body 300 that may be associated with an appropriate medical system. The elongated body includes an articulable portion 304 that may be curved or otherwise bend to a desired angle relative to a base 302 of the articulable portion which may correspond to a non-articulable portion of the elongated body, a rigid portion of the elongated body, or other rigid support that the articulable portion extends distally from. The elongated body may also include a distal end 306. During nominal control, a medical system may control the articulation of the elongated body based on the resulting tip bend angle of the distal end 306. The distal end may be oriented in a direction as indicated by the tip bend angle q. In the depicted embodiment, the distal tip has been articulated such that the distal end is oriented with a tip bend angle of qᵢₙᵢₜᵢₐₗ. For example, the distal end may have been articulated to this orientation and position such that the distal end is oriented towards a target tissue, and thus the bend angle of qᵢₙᵢₜᵢₐₗ is also referred to as a command bend angle.

Referring now to FIG. 3B, after articulating the elongated body 300a via nominal control to the initial tip bend angle qᵢₙᵢₜᵢₐₗ, a tool 308 may be retracted or inserted distally through an internal channel of the elongated body. In one example, an imaging tool that is used to guide navigation to the target tissue during nominal control is retracted from the internal channel and another tool that interacts with the target tissue (e.g., a biopsy tool, an ablation tool, a cutting tool, etc.) is inserted through the internal channel. Forces associated with the retraction or insertion of the tool may also be applied to the elongated body during the relative movement of the tool within the elongated body, such as when the elongated body is controlled to be held in place after the nominal control. The force applied via relative movement of the tool may result in the distal end 306 undesirably shifting in both position and orientation relative to the initial pose of the distal end prior to the relative movement of the tool and elongated body. For example, in the embodiment shown in FIG. 3B, insertion of the tool causes the elongated body 300b to be deformed to a larger radius of curvature with the distal end 306 oriented in a different direction as indicated by tip bend angle qᵢₙₛₑᵣₜ (e.g., despite control being applied to hold the elongated body in place at the command bend angle).

As discussed above, a medical system may control the articulation of the elongated body based on the resulting tip bend angle of the distal end 306 during nominal control. Accordingly, when the elongated body 300b is deformed by tool movement such that the distal tip 306 is oriented with a bend angle of qᵢₙₛₑᵣₜ additional articulation force may be applied to cause the elongated body to be further articulated such that the final tip bend angle q_{final} of the elongated body 300c is parallel (i.e., matches) to the initial tip bend angle qᵢₙᵢₜᵢₐₗ, see FIG. 3C (the inserted tool is not depicted for the sake of clarity). Unless a user separately controls a position of the distal tip during this process, this may result in a translation of the distal tip 306 relative to an original commanded position of the distal tip 306. In other words, the initial and final orientations of the distal tip 306 of the elongated body may be parallel, but the initial and final positions of the of the distal tip 306 may be offset. Specifically, as the stiffness of the elongated body increases during tool insertion, the radius of curvature of the elongated body increases and the bend angle of the distal tip 306 decreases. Correspondingly, the articulation force is increased to force the distal tip back to the desired bend angle. Further, due to the larger increase of the applied articulation force relative to the increased stiffness of the combined tool and elongated body, the resulting radius of curvature of the elongated body 300c after the orientation of the distal tip has been corrected may be smaller than a corresponding radius of curvature of the elongated body 300a prior to insertion of the tool in some embodiments. As such, this type of control strategy that attempts to (e.g., stiffly) hold the elongated body in place during tool insertion or retraction will oppose the insertion and retraction of the tool within the channel of the elongated body. Furthermore, attempting to return the initial commanded pose (e.g., commanded orientation or tip bend angle) is complicated by the increased stiffness of the combined tool and elongated body.

While the above embodiment has been depicted for a tool being inserted into an articulable elongated body, it should be understood that similar changes and corrections for the orientation of the articulated elongated body may occur during other types of relative movement between the tool and elongated body including, for example, retraction of the tool within the elongated body.

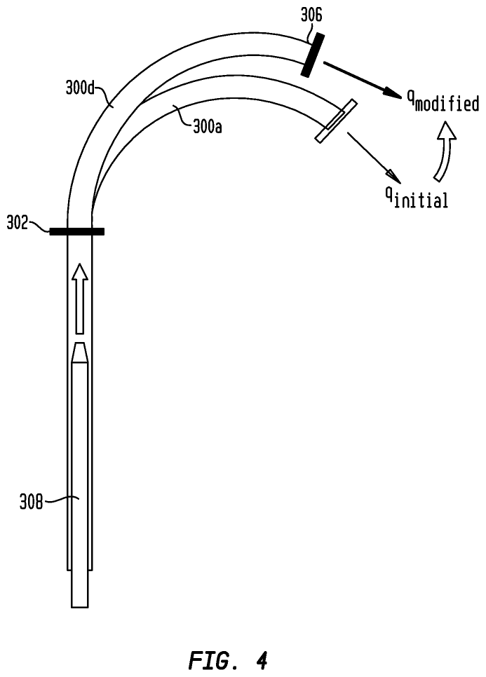

As described previously, a small radius of curvature for the elongated body as well as increased forces being applied to the elongated body and tool during relative movement may result in damage to the tool and/or channel of the elongated body. Accordingly, in some embodiments, an elongated body may be permitted to deviate from a commanded orientation or tip bend angle as set during nominal control during relative movement of a tool and the elongated body. For example, as shown in FIG. 4, the elongated body 300a is initially articulated such that the distal end 306 is oriented with a tip bend angle of qᵢₙᵢₜᵢₐₗ. In some operating modes as detailed further herein, the elongated body is controlled such that it is moved to a more relaxed configuration capable of more easily accommodating relative movement of the tool within the channel of the elongated body. For example, as shown by elongated body 300d the elongated body is controlled such that the distal tip is oriented with a tip bend angle q_{modified} that is less than the initial commanded tip bend angle qᵢₙᵢₜᵢₐₗ. Additionally, the radius of curvature of the elongated body in the second configuration, see 300b, may be greater than the radius of curvature of the elongated body in the initial commanded configuration, see 300a. By controlling the articulable elongated body in such a manner during relative movement of the tool and elongated body, it may be possible to reduce the forces applied to the tool as well as the internal channel and/or lining. Additionally, it may be possible to prevent, or at least reduce, instances in which a tool may be subjected to undesirably small bending radii during relative movement within a channel of an elongated body.

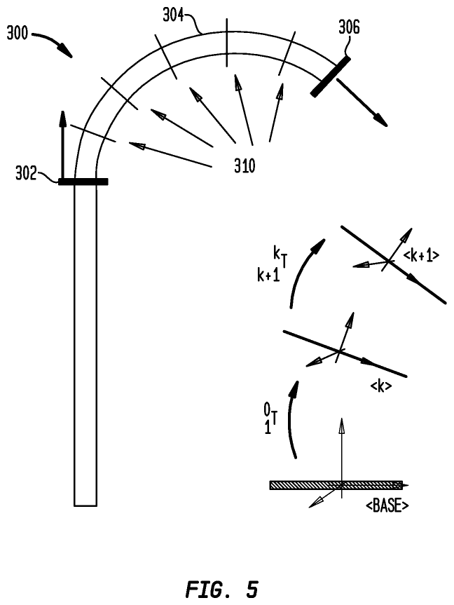

FIG. 5 depicts an articulable elongated body 300 including an articulable portion 304 and associated distal end 306 similar to the embodiments described above. As described previously, the articulable portion of the elongated body may be anchored to a base 302 which may correspond to a non-articulable portion of the elongated body, a rigid portion of the elongated body, and/or other appropriate structure. In some embodiments, the position, orientation, or pose of the base in a reference frame may be known. Additionally, as previously described, a medical system may include one or more sensors that are configured to measure a parameter related to either a shape or curvature of a plurality of sequentially located portions 310 forming the overall articulable portion 304. Thus, by knowing an orientation of each of these intermediate portions relative to each other it may be possible to determine information related to the shape of the overall articulable portion as well as subsections of the articulable portion. In one such embodiment, and as elaborated on further below, an appropriate transform T, which may correspond to a measured angular offset between adjacent portions of the elongated body may be integrated from the base reference frame over the intervening sub-portions of the articulable portion of the elongated body to provide an orientation and/or position of any portion of the articulable portion including the distal end 306. The ability to measure the parameters related to these smaller portions of the articulable elongated body may be beneficial in instances where longer articulable portions of the elongated body are used and/or where the articulable portion of the elongated body may be articulated to follow a tortuous route with different radii of curvature along a length of the elongated body. In such an implementation the overall shape may be computed through integration of the separate segments (i.e., chaining). For example, the orientation of the distal end 306 may be determined by integrating the individual rigid transforms for each segment connecting the distal end 306 to the base 302 which may have a known pose (i.e., position and orientation).

When the orientations of the various segments are known relative to one another it may be possible to determine a local radius of curvature for a plurality of sequentially located portions of the articulable portion of an elongated body. For example, if the individual segments corresponding to the different portions of the elongated body are spaced sufficiently close to one another, the local radius of curvature may be computed using the vertices of the adjacent pairs of segments using the known lengths of the segments d1 and d2, a length d3 corresponding to a distance extending between the two opposing vertices of the segments, and the area A of the triangle enclosed by these segments. For example, the local radius of curvature of the portion of the articulable elongated body extending between two segments <k−1> and <k+1>, or other appropriate segments along the length of the elongated body, may be determined as:

$${r}_{[ k - 1 , k + 1 ]} = \frac{{d}_{1} {d}_{2} {d}_{3}}{4 A}$$

Other methods for determining the local radius of curvature, and/or other appropriate parameters related to the curvature and/or shape of the articulable portion of elongated body, may also be used.

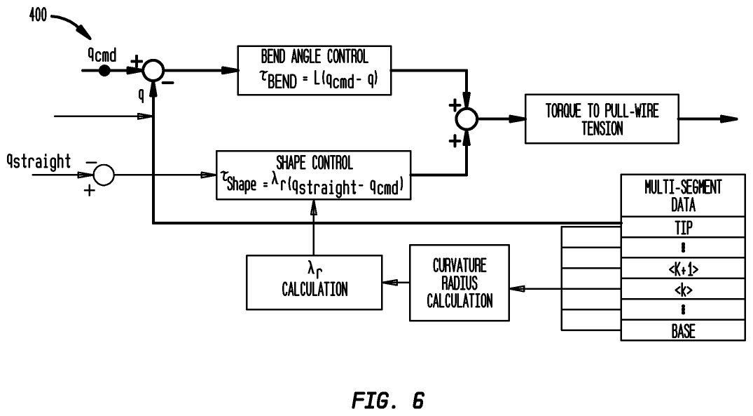

FIG. 6 is block diagram of one embodiment of a control algorithm 400 that may be implemented to control the articulation of one or more articulable portions of an elongated body during the relative movement of a tool positioned within a channel of the elongated body. In the depicted embodiment, the actual bend angle q of a portion of an elongated body may be subtracted from a commanded bend angle q_{cmd}. q_{cmd} may be a bend angle that is manually commanded by a user and/or an associated control system. Correspondingly, the actual bend angle q may be sensed using one or more appropriate sensors. For example, as shown in the depicted embodiment, the relative orientation of a plurality of individual segments of the articulable portion may be measured at Multi-Segment Data block. As noted previously, each of the segments may have an individual reference frame. These reference frames may be appropriately transformed and integrated such that the overall resulting bend angle q may be determined. Regardless of how q is determined, the actual bend angle q may be subtracted from the commanded bend angle q_{cmd} to provide an error term qₑᵣᵣ which may physically correspond to the offset between the commanded and actual tip bend angle. This error term may be input to the bend angle control block where it is multiplied by an appropriate gain L to provide a corrected motor torque to be applied to a pull wire used to control the articulable portion according to the equation:

τ_{bend}=L(q_{cmd}−q)

The torque τₛₕₐₚₑ to articulate the elongated body to the commanded tip bend angle is determined based on a difference between the commanded bend angle q_{cmd} and unarticulated bend angle q_{straight} of the articulated portion multiplied by an appropriate gain factor λᵣ, which is detailed further below, according to the formula:

τₛₕₐₚₑ=λᵣ(q_{straight}−q_{cmd})

These two torques may be combined to provide the commanded torque for outputting to the one or more actuators used to apply tension to the associated tetehers or other structures used to articulated the elongated body. This output combined torque may be defined as follows:

$$τ = L ( {q}_{cmd} - q ) + {λ}_{r} ( {q}_{straight} - {q}_{cmd} ) = L ( \frac{L - {λ}_{r}}{L} {q}_{cmd} + \frac{{λ}_{r}}{L} {q}_{straight} - q )$$

These above two processes may be implemented during both nominal operation and during relative movement of a tool through a channel of the elongated body. The differences in operation between these two operating modes is defined by the gain factor kr. Again referencing the Multi-Segment Data block, the measurements of the bend angles, or other appropriate parameter, of the individual segments of the articulable portion of the elongated body may be input into a Curvature Radius Calculation block which may determine a radius of curvature of one or more portions of the articulable portion of the elongated body. While the radius of curvature is determined in the specific embodiment, other appropriate parameters related to a shape of the articulated portion of the elongated body may also be used as the disclosure is not so limited. In either case, the resulting determined parameter, i.e. the radius of curvature, may be output to the λᵣ Calculation block where λᵣ may be determined according to the following control law:

$${λ}_{r} = \{ \begin{matrix}0 & when {min}_{k} ( r ) > {r}_{thr} \\ L ( {r}_{thr} - {min}_{k} ( r ) ) & otherwise\end{matrix}$$

In the above control law, λᵣ is zero when a minimum radius of curvature across all of the different portions of the articulable portion of the elongated body is greater than a threshold radius of curvature. In other embodiments, this may also correspond to a threshold for an average radius of curvature of the articulable portion of the elongated body. This may either be an average over the entire articulable portion of the elongated body and/or the minimum radius may be computed as a moving average filter along the articulable portion of the elongated body where the width of the moving average filter may be chosen to reduce sensitivity of the method to high spatial frequency noise.

In instances in which the determined parameter is less than or equal to, or otherwise lies outside of a desired range of, the threshold, λᵣ may be determined based on a difference between the measured parameter and threshold parameter. In the depicted embodiment, λᵣ in such an operating mode is equal to the gain factor L multiplied by a difference between the threshold radius of curvature and the measured minimum radius of curvature.

Based on the above control law, when the parameter is greater than the threshold parameter, the shape control may result in a τₛₕₐₚₑ of 0. This may correspond to the articulable portion of the elongated body having a large enough radius of curvature such that changing the operation of the system based on tool insertion may not be needed. Correspondingly, when the threshold parameter is met (i.e., the parameter is less than or equal to the threshold radius), the gain becomes a function of both the threshold radius and the measured radius of curvature. Specifically, as the minimum radius of curvature decreases, λᵣ increases resulting in an overall decrease in the combined output torque τ, see formula above. This combined control law may help to address a desire for less error in commanded orientation of an articulable portion while also permitting increased radii of curvature during movement of a tool when a commanded radius of curvature, bend angle, or other parameter may be outside of a desired operating range of the tool. The amount of change in operation may also increase as the deviation from a desired operating range increases.

In some embodiments, the control strategy may revert to a nominal control strategy after relative movement of the tool has terminated as determined by a sensor, user input, or other appropriate input (e.g., after the tool is fully inserted into or removed from the elongated body). For example, in some embodiments, this may include stopping controlling the articulable portion of an elongated body based at least in part on a parameter related to a curvature of one or more portions of the elongated body as described herein. Upon stopping this control strategy, control of the articulable portion of the elongated body may be based on another other appropriate parameter including, for example, a position and/or bend angle of a distal tip of the elongated body. This control strategy may either be the same or different from the control strategy applied prior to controlling the articulable portion based on the parameter related to the curvature of the one or more portions of the elongated body. In some instances, to avoid jumps in the operation of the articulable elongated body, a time decay may be applied when transitioning between control strategies in some embodiments.

While the control algorithm illustrated in the block diagram of FIG. 6 has been described relative to motor torques used actuators to apply tension to one or more tethers used to control articulation of the elongated body, it should be understood that any other appropriate control parameter for controlling articulation of the elongated body may be used as well. For example, appropriate parameters for controlling articulation of the elongated body may include, but are not limited to, tension, force, motor current, and/or any other appropriate parameter that may be used to control an actuator configured to control articulation of an elongated body.

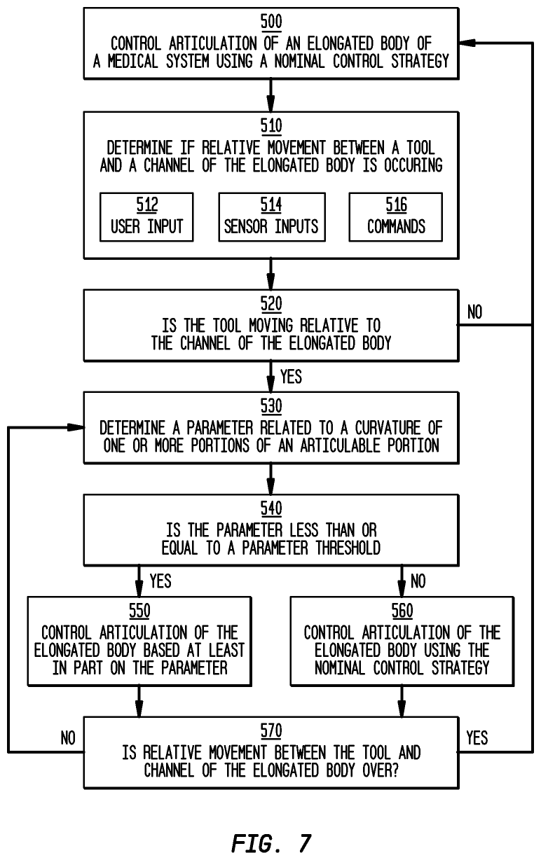

FIG. 7 depicts one embodiment of a method that may be used to control articulation of an elongated body. In some embodiments, the method may start at 500 where the articulation of an elongated body of a medical system may be controlled using a first nominal control strategy used during nominal operation of the medical system. At 510, it may be determined if relative movement between a tool in a channel of the elongated body of the medical system is occurring or will occur, such as relative movement of the tool through an articulable portion of the elongated body. For example, as noted previously, a tool may be inserted through, retracted from, or otherwise moved relative to the channel of the elongated body during various medical procedures. This determination may be provided in any appropriate manner including, for example, input from a user at 512, sensors configured to detect relative movement of a tool positioned within the channel of the elongated body at 514, commands from a user and/or a control system of the medical system to move the tool relative to the channel at 516, combinations of the foregoing, and/or any other appropriate method of determining the noted relative movement of the tool.

At step 520, if the tool is not moving (or will not move) relative to the channel of the elongated body, the depicted process may continue operating using the nominal control strategy of 500. However, if the tool is moving (or will move) relative to the channel of the elongated body, the process may continue on to step 530 where a different control strategy in a second operating mode may be implemented. That said, in some embodiments, the depicted systems and methods may be operated using this second mode of operation even when relative movement between the tool and channel of the elongated body is not occurring. In some embodiments, the relative movement of the tool and the elongated body of interest for changing control strategy may include movements of the tool that traverse the articulable portion of the elongated body. This may include insertion or removal of the tool. In some embodiments, other movements, such as movements associated with performing biopsy by a biopsy tool or ablation by an ablation tool after these tools have been inserted may not change the control strategy. For these movements, the elongated body position, radius of curvature, and/or a tip bend angle may be held to facilitate interaction with the desired target tissue location.

At step 530, a parameter related to a curvature of one or more portions of an articulable portion of the elongated body may be determined. As noted previously, this parameter may correspond to a radius of curvature of one or more portions of the articulable portion in some embodiments and may be determined with one or more sensed parameters related to a shape of the elongated body. Additionally, this parameter may either be a parameter corresponding to the entire articulable portion or the parameter may be determined for a plurality of portions of the articulable portion. This parameter may be determined in any appropriate manner, including the methods described above relative to the use of shape sensors and/or other sensors configured to sense a parameter related to the curvature or shape of the articulable portion of the elongated body.

At step 540, the parameter may be compared to a parameter threshold. For example, in some embodiments, a minimum radius of curvature, an average radius of curvature, or other appropriate characterization of the radius of curvature of the articulable portion of the elongated body may be compared to a threshold radius of curvature. If the parameter is greater than the parameter threshold, or otherwise within a desired operating range of the parameter, the medical system may continue to control articulation of the elongated body using a strategy similar to, or the same as, the nominal control strategy as indicated at step 560. However, if the parameter is less than or equal to the parameter threshold, or otherwise outside a desired operating range of the parameter, the medical system may control articulation of the elongated body based at least in part on the determined parameter at step 550. One example of how such a control strategy may be implemented as described above relative to FIG. 6.

While any appropriate type of control strategy may be implemented at step 550, in some embodiments, the one or more actuators used to control articulation of the elongated body may be controlled to provide various modifications to the commanded articulation. Specifically, the actuators may be controlled to decrease (or otherwise change) a commanded bend angle of the articulable portion during relative movement of the tool as compared to a corresponding commanded bend angle of the articulable portion during nominal operation. For example, the commanded bend angle may be reduced when the radius of curvature of one or more portions of articulated elongated body is less than or equal to a threshold radius of curvature. This may result in an increase in the radius of curvature of the articulable portion of the elongated body in some embodiments. In yet another embodiment, the actuators may be controlled to decrease the commanded bend angle by reducing a tension applied to at least one flexible tether operatively coupled to the articulable portion of the elongated body. Of course, while several possible actions are detailed above for use during control of the articulable portion during relative movement of a tool and the elongated body, it should be understood that other types of control strategies and operations may be implemented in some embodiments as the disclosure is not so limited.

At step 570, it may be determined whether or not the relative movement between the tool and channel of the elongated body is over. In other words, it may be determined if the tool is stationary relative to the elongated body. For example, it may be determined that a tool is fully inserted or retracted to determine that the relative movement is over. If the relative movement is not over, the process may return to step 530. Alternatively, if the relative movement is over, the process may return to step 500. Again, this may include determining that a tool is either fully inserted or retracted from the elongated body. Thus, the system may control articulation of the elongated body such that the elongated body returns to a commanded configuration, i.e. bend angle, when the relative movement of the tool is over. If a stiffness of the combined assembly of the elongated body and tool is different from an initial stiffness of the elongated body, a position of the distal tip of the elongated body may be different from an initial position of the distal tip of the elongated body. In such instances, a user may need to reposition the distal tip of the elongated body to continue with the desired procedure.

It should be understood that the above described methods and control laws are exemplary. Thus, any appropriate control law including different relationships than those described herein may be used to provide the desired functionality as the disclosure is not so limited. Additionally, the various steps in the algorithms and methods disclosed herein may be arranged in different orders, may include various additional intermediate steps, omit certain steps, and/or have other changes relative to those disclosed herein in as the disclosed methods and system are not limited in this fashion.

In some embodiments, the various methods and control algorithms disclosed above, and elsewhere herein, may be implemented in software to execute on a processor of a computing system, including on a processor of the control system 112 of FIG. 1 or other appropriate medical system. When implemented in software, the elements of these embodiments may be essentially the code segments to perform the disclosed tasks. The program or code segments can be stored in a processor readable storage medium or device that may have been downloaded by way of a computer data signal embodied in a carrier wave over a transmission medium or a communication link. The processor readable storage device may include any medium that can store information including an optical medium, semiconductor medium, and magnetic medium. Processor readable storage device examples include an electronic circuit, a semiconductor device, a semiconductor memory device, a read only memory (ROM), a flash memory, an erasable programmable read only memory (EPROM), a floppy diskette, a CD-ROM, an optical disk, a hard disk, or other storage device. The code segments may be downloaded via computer networks such as the Internet, Intranet, etc. Thus, in some embodiments at least one non-transitory computer-readable medium may have instructions thereon that, when executed by at least one processor (e.g., the processor of any of the systems disclosed herein), may perform any one of the methods disclosed herein.

While several examples of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be illustrative and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific examples of the disclosure described herein. It is, therefore, to be understood that the foregoing examples are presented by way of example only and that, within the scope of the appended claims and equivalents thereto, the disclosure may be practiced other than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

1. A medical system comprising:
an elongated body including a channel extending through the elongated body, wherein the elongated body includes an articulable portion extending along at least a portion of a length of the elongated body;
one or more actuators operatively coupled to the articulable portion of the elongated body by one or more flexible tethers, wherein the one or more actuators are configured to apply a tension to the one or more flexible tethers to articulate the articulable portion of the elongated body;
one or more sensors configured to sense a first parameter related to a shape of the articulable portion of the elongated body; and
a processor operatively coupled with the one or more actuators and the one or more sensors, wherein the processor is configured to:
determine a second parameter related to a curvature of one or more portions of the articulable portion based at least in part on the sensed first parameter; and
control the one or more actuators based at least in part on the determined second parameter.

2. The medical system of claim 1, wherein the processor is configured to control the one or more actuators based on the determined second parameter during relative movement between the channel of the elongated body and a tool disposed in the channel.

3. The medical system of claim 2, wherein the processor is configured to determine if the relative movement of the tool and the channel is occurring.

4. The medical system of claim 2, wherein the processor is configured to control the one or more actuators to decrease a bend angle of the articulable portion during relative movement of the tool and the channel.

5. The medical system of claim 2, wherein the processor is configured to return the articulable portion to a commanded bend angle when the tool is fully inserted or removed from the elongated body.

6. The medical system of claim 1, wherein the determined second parameter includes a tip bend angle of the one or more portions of the articulable portion.

7. The medical system of claim 1, wherein the determined second parameter includes a radius of curvature of the one or more portions of the articulable portion.

8. The medical system of claim 7, wherein the one or more portions of the articulable portion are a plurality of portions of the articulable portion.

9. The medical system of claim 7, wherein the radius of curvature is a minimum radius of curvature of the one or more portions of the articulable portion.

10. The medical system of claim 7, wherein:
the radius of curvature is a minimum radius of curvature of the one or more portions of the articulable portion; and
the processor is configured to control the one or more actuators based at least in part on the radius of curvature and a threshold radius of curvature.

11. The medical system of claim 1, wherein the determined second parameter includes a tip bend angle of the one or more portions of the articulable portion.

12. The medical system of claim 1, wherein the one or more sensors includes a shape sensor.

13. The medical system of claim 1, wherein:
the processor is configured to control the actuators based on the determined second parameter during insertion or retraction of a tool through the articulable portion of the elongated body; and
the control of the actuators causes at least one of:
an increase in a radius of curvature of the one or more portions of the articulable portion; and
a decrease in a bend angle of the one or more portions of articulable portion.

14. A method for controlling articulation of a medical system, the method comprising:
determining a parameter related to a curvature of one or more portions of an articulable portion of an elongated body including a channel extending through the elongated body; and
controlling articulation of the articulable portion based at least in part on the determined parameter.

15. The method of claim 14, further comprising:
determining if a relative movement of a tool disposed in the channel and the channel is occurring; and
controlling the articulation of the articulable portion based at least in part on the determined parameter during relative movement between the channel of the elongated body and a tool disposed in the channel.

16. The method of claim 15, wherein controlling the articulation of the articulable portion includes decreasing a commanded bend angle of the articulable portion during relative movement of the tool and the channel; and
the method further comprises returning the articulable portion to a commanded bend angle when the tool is fully inserted into or removed from the elongated body.

17. The method of claim 16, wherein the parameter includes a radius of curvature of the one or more portions of the articulable portion.

18. The method of claim 17, wherein the one or more portions of the articulable portion are a plurality of portions of the articulable portion and the radius of curvature is an average radius of curvature of the plurality of portions of the articulable portion.

19. The method of claim 17, wherein the radius of curvature is a minimum radius of curvature of the one or more portions of the articulable portion.

20. The method of claim 17, wherein controlling articulation of the articulable portion includes controlling articulation of the articulable portion based at least in part on the radius of curvature and a threshold radius of curvature.

21. A non-transitory computer-readable medium comprising instructions thereon that, when executed by at least one processor, perform operations for controlling articulation of a medical system, the operations comprising:
determining a parameter related to a curvature of one or more portions of an articulable portion of an elongated body including a channel extending through the elongated body; and
controlling articulation of the articulable portion based at least in part on the determined parameter.
